(19) United States
(12) Patent Application Publication
(10) Pub. No.: US 2019/0175263 A1
(43) Pub. Date: Jun. 13, 2019

(54) BALLOON CATHETER WITH REVERSE SPIRAL GUIDEWIRE

(71) Applicant: Biosense Webster (Israel) Ltd., Yokneam (IL)

(72) Inventors: Andres Claudio Altmann, Haifa (IL); Assaf Govari, Haifa (IL)

(73) Assignee: Biosense Webster (Israel) Ltd., Yokneam (IL)

(21) Appl. No.: 15/838,962

(22) Filed: Dec. 12, 2017

(51) Int. Cl. A61B 18/14; A61M 25/01; A61M 25/10

(52) U.S. Cl. CPC A61B 18/1492; A61M 25/0119; A61M 2025/0063; A61M 2025/1056; A61M 25/1002; A61M 25/1018

(57) ABSTRACT

An apparatus includes a shaft for insertion into a heart, a reverse-lasso catheter disposed at the distal end of the shaft, and multiple sensing-electrodes. The shaft has a distal end and a proximal end. The distal end of the shaft defines a longitudinal axis. The reverse-lasso catheter includes a base segment, and a spiral segment that retrogrades in the proximal direction around the base segment as the spiral segment spirals about the longitudinal axis. The sensing-electrodes are disposed over the spiral segment and are configured to obtain cardio-electrograms.

### FIELD OF THE INVENTION

The present invention relates generally to medical probes, and particularly to cardiac ablation catheters.

### BACKGROUND OF THE INVENTION

Pulmonary vein isolation procedures often apply ablation to induce circumferential lesions at an ostium of a pulmonary vein for the purpose of eliminating unwanted electrical pathways that may cause arrhythmia. For example, U.S. Patent Application Publication 2016/0175041 describes cardiac ablation that is carried out by introducing a catheter into the left atrium, extending a lasso guide through the lumen of the catheter to engage the wall of a pulmonary vein, and deploying a balloon over the lasso guide. The balloon has an electrode assembly disposed its exterior. The electrode assembly includes a plurality of ablation electrodes circumferentially arranged about the longitudinal axis of the catheter. The inflated balloon is positioned against the pulmonary vein ostium, so that the ablation electrodes are in galvanic contact with the pulmonary vein, and electrical energy is conducted through the ablation electrodes to produce a circumferential lesion that circumscribes the pulmonary vein.

As another example, U.S. Patent Application Publication 2013/0006238 describes a catheter that includes an elongated body, a distal assembly with a shape-memory member defining a generally circular form, and a control handle adapted to actuate a deflection puller wire for deflecting a portion of the elongated body, and a contraction wire for contracting the generally circular form. In a more detailed embodiment, the catheter has a distal assembly with a helical form or a crescent form carrying a plurality of irrigated ablation ring electrodes and a plurality of smaller ring electrodes adapted for impedance recording or pulmonary vein potential recording.

U.S. Patent Application Publication 2016/0338769 describes a catheter that has an electrode assembly adapted for use at an ostium and a stabilizing assembly movable between a stowed position within the catheter and a deployed position in the tubular region of the ostium. The stabilizing assembly has a balloon member that can be extended distally into the tubular region and inflated to wedge itself in the tubular region for anchoring the electrode assembly. The balloon member is mounted on an elongated extender that extends through the catheter and is provided with longitudinal movement to advance and withdraw the balloon between the stowed and deployed positions.

U.S. Pat. No. 7,285,119 describes a catheter body that includes a proximal portion, an intermediate portion extending from the proximal portion and defining a longitudinal axis, and a distal portion extending from the intermediate portion and terminated by a distal end of the catheter body; the distal portion forms a coil about a central loop axis, which is substantially parallel to the longitudinal axis. A lumen extends through the proximal and intermediate portions of the catheter body and is terminated at an opening proximal to an ablation section, which is formed along the coil. In an embodiment, an inflated balloon is provided as to assist in centering an ablation loop about the pulmonary vein ostium.

### SUMMARY OF THE INVENTION

An embodiment of the present invention provides an apparatus including a shaft for insertion into a heart, a reverse-lasso catheter disposed at the distal end of the shaft, and multiple sensing-electrodes. The shaft has a distal end and a proximal end. The distal end of the shaft defines a longitudinal axis. The reverse-lasso catheter includes a base segment, and a spiral segment that retrogrades in the proximal direction around the base segment as the spiral segment spirals about the longitudinal axis. The sensing-electrodes are disposed over the spiral segment and are configured to obtain cardio-electrograms.

In some embodiments, the spiral segment has a first end that is connected to the base segment, and a second end that is free and is closer to the proximal end than the first end, along the longitudinal axis.

In some embodiments, the apparatus includes an inflatable balloon disposed over a portion of the distal end of the shaft. The inflatable balloon has a plurality of radiofrequency ablation electrodes arranged circumferentially over an external surface of the inflatable balloon. In an embodiment, the inflatable balloon is configured to perform pulmonary vein isolation by applying radiofrequency ablation. In another embodiment, the reverse-lasso catheter is configured to engage an interior wall of a pulmonary vein.

There is additionally provided, in accordance with an embodiment of the present invention, a method including inserting a shaft into a heart, the shaft having a distal end and a proximal end. The distal end of the shaft defines a longitudinal axis. A reverse-lasso catheter, which includes a base segment and a spiral segment that retrogrades in the proximal direction around the base segment as the spiral segment spirals about the longitudinal axis, is advanced to engage an interior wall of the heart. Cardio-electrograms are obtained using a plurality of sensing-electrodes disposed over the spiral segment.

There is also provided, in accordance with an embodiment of the present invention, a method of manufacturing a reverse-lasso catheter including providing a shaft having a distal end and a proximal end. The distal end of the shaft defines a longitudinal axis. A reverse-lasso catheter, which includes a base segment and a spiral segment that retrogrades in the proximal direction around the base segment as the spiral segment spirals about the longitudinal axis, is disposed at the distal end of the shaft. Cardio-electrograms are obtained using a plurality of sensing-electrodes disposed over the spiral segment.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a catheter-based position-tracking and ablation system, in accordance with an embodiment of the present invention;

FIG. 2 is a schematic, pictorial illustration of the distal portion of the catheter shown in FIG. 1 in an operating position for ablation, in accordance with an embodiment of the invention; and

FIG. 3 is a flow chart that schematically illustrates a method of pulmonary vein isolation, in accordance with an embodiment of the invention.

### DETAILED DESCRIPTION OF EMBODIMENTS

### Overview

Embodiments of the present invention that are described herein provide improved cardiac ablation catheters and associated methods. Some embodiments in particular, provide apparatus and method for acquiring cardio-electrograms in-situ immediately before and after ablation, which may serve as a useful diagnostic tool for confirming the functional isolation of a pulmonary vein.

In some embodiments, a cardiac catheter comprises an inflatable balloon with ablation electrodes fitted at a distal end of a shaft, and a lasso catheter connected to the shaft distally to the balloon. The lasso catheter comprises a guidewire having a straight base segment and a lasso spiral segment. The guidewire is used for guiding and positioning the balloon at the ostium of a pulmonary vein. In addition, the spiral segment of the guidewire comprises a plurality of sensing-electrodes disposed thereon, for acquiring cardio-electrograms around the wall of the pulmonary vein, in vicinity to the ostium.

In some embodiments, the spiral segment of the guidewire is configured to spiral backwards, around the straight base segment, i.e., in ‘reverse,’ towards the ablation balloon, which as described above is fitted just proximally to the guidewire.

By having the spiral segment of the lasso catheter retrograding proximally over the straight base segment, the sensing-electrodes disposed over the spiral segment are brought closer to the tissue being ablated, for example to a vicinity of the ostium of the pulmonary vein.

Consequently, as the reverse spiral segment of the guidewire engages the wall of the pulmonary vein and the inflated balloon is in firm contact with the ostium of the pulmonary vein, electrograms are measured closer to the ostium of the pulmonary vein than would be possible with a spiral segment that spirals in the distal direction. This configuration is feasible even in an otherwise disrupting presence of a long distal tip of the balloon.

In the description hereinafter, for simplicity, the disclosed lasso catheter configuration, i.e., spiraling in a reverse direction, retrograding in the proximal direction around its base segment, is referred to as ‘reverse-lasso catheter.’

In optional embodiments, various diagnostic and/or treating devices, other than an ablation balloon, may be fitted on a portion of a distal end of a shaft of a catheter, just proximally to the lasso catheter. The otherwise physically disrupting presence of elements of such devices no longer pose the same constrains when designing a reverse-lasso catheter.

The disclosed sensing apparatus and technique for obtaining cardio-electrograms has thus the distinct advantage that they can collect more accurate and detailed measurements of the electrical activity at the ostium of the pulmonary vein. The disclosed configuration thus enables the physician to prepare a better treatment plan before performing an ablation, and to better confirm in-situ (i.e., while the balloon is still available for performing corrective ablation, if required) the degree of elimination of the unwanted electrical pathways. The disclosed technique thus improves the probability that an ablation treatment will prevent recurring erroneous electrical activity which might have led to a recurring arrhythmia.

### System Description

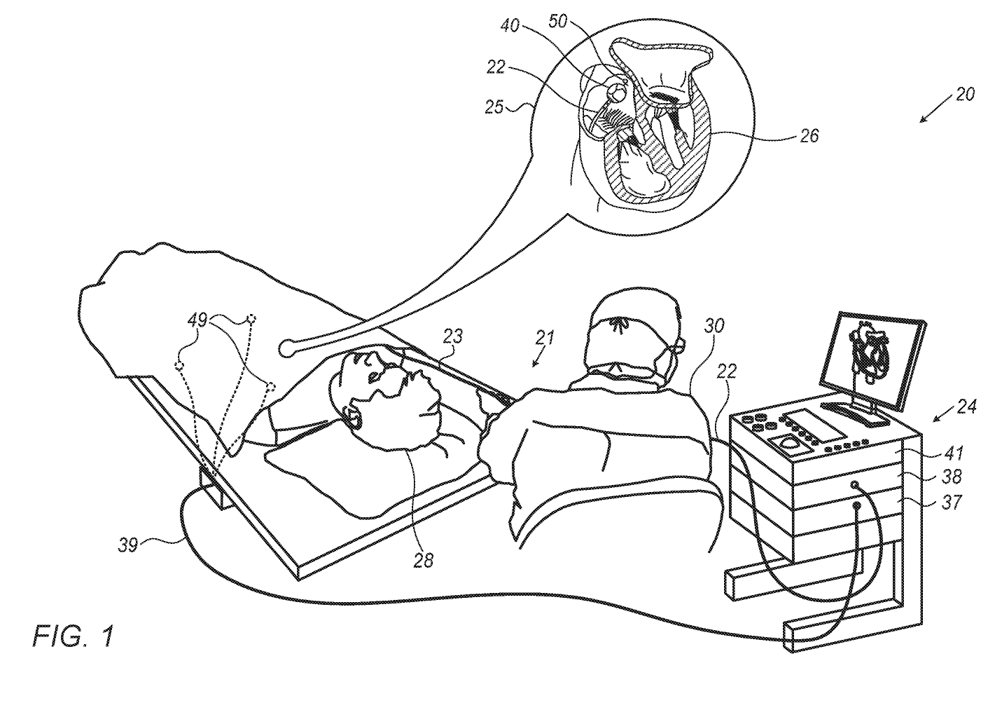

FIG. 1 is a schematic, pictorial illustration of a catheter-based position-tracking and ablation system 20, in accordance with an embodiment of the present invention. System 20 comprises a catheter 21, wherein a shaft 22 of the catheter is inserted into a heart 26 of a patient 28 through a sheath 23. The proximal end of catheter 21 is connected to a console 24.

Console 24 comprises a control unit 38 for receiving signals from catheter 21, as well as for applying RF energy via catheter 21 to ablate tissue in a left atrium of heart 26. Control unit 38 is further configured for controlling other components of system 20. In the embodiment described herein, catheter 21 may be used for any suitable therapeutic and/or diagnostic purposes, such as electrical sensing and/or ablation of an ostium tissue of a pulmonary vein in a left atrium of heart 26.

A physician 30 inserts shaft 22 through the vascular system of patient 28. As seen in inset 25, an inflatable balloon 40 and a reverse-lasso catheter 50 for sensing are both fitted at the distal end of shaft 22. During the insertion of shaft 22, balloon 40 and reverse-lasso catheter 50 are maintained in a collapsed configuration inside sheath 23. By containing balloon 40 and reverse-lasso catheter 50 in a collapsed configuration, sheath 23 also serves to minimize vascular trauma along the way to target location. Physician 30 navigates the distal end of shaft 22 to a target location in heart 26. Once the distal end of shaft 22 has reached the target location, physician 30 retracts sheath 23, letting reverse-lasso catheter 50 expand. Physician 30 then manipulates shaft 22 to have reverse-lasso catheter 50 engage an interior wall of a pulmonary vein. Physician 30 then inflates balloon 40 disposed over a portion of the distal end of shaft 22 and further manipulates shaft 22 to bring balloon 40 to contact with the ostium of the pulmonary vein.

Console 24 comprises a processor 41, typically a general-purpose computer, with suitable front end and interface circuits 37 for receiving signals from external-electrodes 49, which are typically placed around the chest of patient 26. For this purpose, processor 41 is connected to external-electrodes 49 by wires running through a cable 39.

Processor 41 is typically programmed in software to carry out the functions described herein. The software may be downloaded to the computer in electronic form, over a network, for example, or it may, alternatively or additionally, be provided and/or stored on non-transitory tangible media, such as magnetic, optical, or electronic memory.

Although the pictured embodiment relates specifically to the use of a sensing lasso and a balloon for ablation of heart tissue, the elements of system 20 and the methods described herein may alternatively be applied in controlling ablation using other sorts of multi-electrode sensing and/or ablation devices, such as multi-arm sensing/ablation catheters.

### Balloon Catheter with Reverse Spiral Guidewire

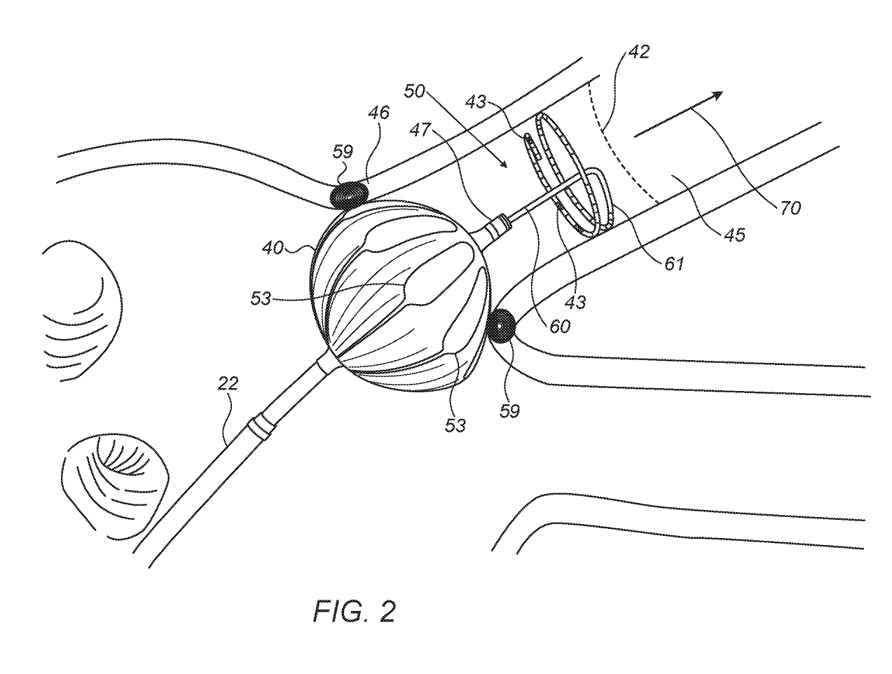

FIG. 2 is a schematic, pictorial illustration of the distal portion of the catheter shown in FIG. 1 in an operating position for ablation, in accordance with an embodiment of the invention. The distal end of shaft 22 defines a longitudinal axis 70 along and parallel to the distal end of shaft 22.

As seen, reverse-lasso catheter 50 comprises a straight base segment 60 and a spiral guidewire 61. As also seen, spiral guidewire 61 retrogrades in the proximal direction around base segment 60 of reverse-lasso catheter 50, while spiraling about longitudinal axis 70. In other words, spiral guidewire 61 has a first end that is connected to base segment 60, and a second end that is free and is closer to the proximal end of the catheter than the first end, along the longitudinal axis.

Reverse-lasso catheter 50 is deployed beyond a distal tip 47 of inflated balloon 40, and extends using base segment 60 further distally so as to engage the interior wall of a pulmonary vein 45. As seen, inflated balloon 40 closes off pulmonary vein 45 at an ostium 46. Balloon 40 has multiple ablation electrodes 53 disposed on its external surface. In a pulmonary-vein isolation procedure, passage of electrical energy through the electrodes 53 creates a circumferential lesion 59 at ostium 46 that blocks electrical propagation and isolates the pulmonary vein from the heart.

In an embodiment, reverse-lasso catheter 50 is made at least partially of a shape memory alloy having a self-configurable pre-formed shape comprising straight base segment 60 and spiral guidewire 61 parts. Thus, when physician 30 retracts sheath 23 reverse-lasso catheter 50 self-configures itself from a collapsed straight configuration into an expanded configuration having a straight base segment 60 and a spiral guidewire 61 parts. In another embodiment, the guidewire is made at least partially of a shape memory alloy having pre-formed shape comprising straight base segment 60 and spiral guidewire 61 parts. Reverse-lasso catheter 50 is electrically wired for providing a heating electrical current to the guidewire, so as to cause, when heated, to the guidewire to configure itself into the pre-formed shape.

Multiple ring sensing-electrodes 43 are disposed over spiral guidewire 61. Sensing-electrodes 43 are useful for obtaining electrograms to confirm electrical isolation of the pulmonary vein following ablation while reverse-lasso catheter 50 still engaging the wall of pulmonary vein 45.

As seen in FIG. 2, sensing-electrodes 43 are located closer to ostium 49 relative to possible positions 42 of that might be achieved using alternative lasso configurations, such as forward spiraling ones. In other words, a reverse lasso design prevents sensing-electrodes 43 from being unavoidably located deeper into pulmonary vein 45. Indeed, as noted above, sensing electrodes 43 can be brought closer to ostium 49 and specifically in vicinity to circumferential lesion 59. The close proximity of the electrodes to the ablation site improves the likelihood that the electrograms acquired by sensing electrodes 43 will accurately assess the degree of pulmonary vein 45 isolation.

The example illustration shown in FIG. 2 is chosen purely for the sake of conceptual clarity. Other geometric configurations for ablation electrodes 53 are possible, for example a spiral arrangement, or concentric rings. Any type of the sensing electrodes may be fitted over the lasso, i.e., other than the exemplified ring sensing-electrodes 43. In alternative embodiments, sensing electrodes 43 can be brought further closer to balloon 40, for example by increasing the spiral period or by adding more windings to segment 61. Other type of sensing and/or ablation geometries, such as a multiple-ray catheter (e.g., the Pentaray® Catheter, produced by Biosense Webster, Inc.) may also adopt a reverse geometry.

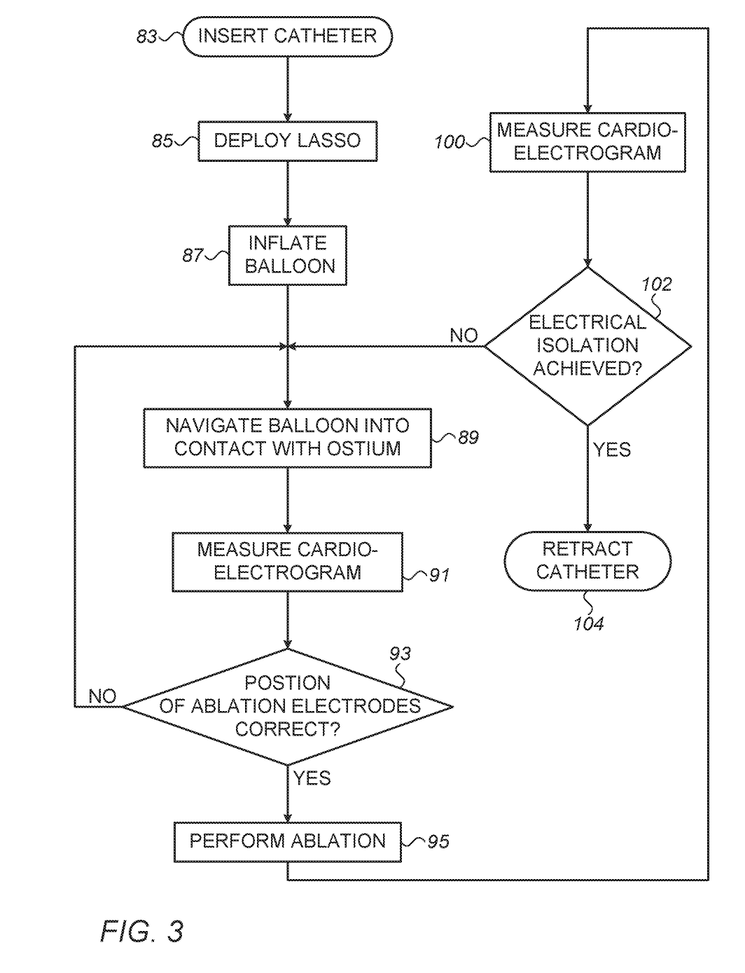

FIG. 3 is a flow-chart that schematically illustrates a method of pulmonary vein isolation, in accordance with an embodiment of the invention. The procedure may begin with physician 30 inserting a cardiac catheter into the left atrium of a heart, at an insertion step 83.

Next, at a deployment step 85, physician 30 deploys and positions reverse-lasso catheter 50 to engage the interior wall of pulmonary vein 45. Next, at an inflation step 87, physician 30 inflates balloon 40. At a positioning step 89, physician 30 brings balloon 40 in into circumferential contact with ostium 46 of pulmonary vein 45 so as to occlude ostium 46. Once reverse-lasso catheter 50 and balloon 40 are in position, pre-ablation cardio-electrograms are acquired by physician 30, at a measurement step 91.

The method now proceeds to a decision step 93, in which physician 30 determines based on the electrograms whether ablation electrodes 53 are correctly positioned. If the determination at decision step 93 is negative, then the method returns to step 89 and physician 30 may reattempt to optimally position balloon 40.

If the determination at decision step 93 is affirmative, then the method proceeds to an ablation step 95 in which physician 30 performs ablation using ablation electrodes 53. The ablation operation creates circumferential lesion 59 in a region of tissue that circumscribes ostium 46. Lesion 59 should block electrical propagation and effectively electrically isolate the pulmonary vein 45 from the heart. In order to confirm functional isolation of pulmonary vein 45, post-ablation electrograms are obtained by physician 30, from sensing-electrodes 43 of reverse-lasso catheter 50, at measurement step 100.

If analysis of the cardio-electrograms, at a decision step 102, shows that lesion 59 does not fully electrically isolate pulmonary vein 45 from the heart, then physician may perform another attempt by looping back to positioning step 89.

After completion of the ablation, the procedure may be iterated for treating another pulmonary vein ostium by withdrawal of the distal end of shaft 22 (i.e., of balloon 40 and reverse-lasso catheter 50). The method may then return to step 85. Alternatively, physician 30 may end the procedure and retract catheter 21 from the heart, at a retraction step 104.

The example flow chart shown in FIG. 3 is chosen purely for the sake of conceptual clarity. In alternative embodiments, additional steps may be performed, such as an injection of contrast agent followed by fluoroscopy imaging. Contact force sensing may also be applied for monitoring the quality of positioning prior to ablation. Additionally, irrigation may be used during ablation, for cooling the ostium tissue.

Although the embodiments described herein mainly address pulmonary vein isolation, the methods and systems described herein can also be used in other applications, such as to measure signals around interior walls of the Inferior Vena Cava (IVC) and/or the Superior Vena Cava (SVC).

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. An apparatus, comprising:
a shaft for insertion into a heart, the shaft having a distal end and a proximal end, wherein the distal end of the shaft defines a longitudinal axis;
a reverse-lasso catheter disposed at the distal end of the shaft, wherein the reverse-lasso catheter comprises a base segment, and a spiral segment that retrogrades in the proximal direction around the base segment as the spiral segment spirals about the longitudinal axis; and
multiple sensing-electrodes, which are disposed over the spiral segment and are configured to obtain cardio-electrograms.

2. The apparatus according to claim 1, wherein the spiral segment has a first end that is connected to the base segment, and a second end that is free and is closer to the proximal end than the first end, along the longitudinal axis.

3. The apparatus according to claim 1, and comprising an inflatable balloon disposed over a portion of the distal end of the shaft, wherein the inflatable balloon has a plurality of radiofrequency ablation electrodes arranged circumferentially over an external surface of the inflatable balloon.

4. The apparatus according to claim 3, wherein the inflatable balloon is configured to perform pulmonary vein isolation by applying radiofrequency ablation.

5. The apparatus according to claim 1, wherein the reverse-lasso catheter is configured to engage an interior wall of a pulmonary vein.

6. A method, comprising:
inserting a shaft into a heart, the shaft having a distal end and a proximal end, wherein the distal end of the shaft defines a longitudinal axis;
advancing a reverse-lasso catheter, disposed at the distal end of a shaft, to engage an interior wall of the heart, wherein the reverse-lasso catheter comprises a base segment and a spiral segment that retrogrades in the proximal direction around the base segment as the spiral segment spirals about the longitudinal axis; and
obtaining cardio-electrograms using a plurality of sensing-electrodes disposed over the spiral segment.

7. The method according to claim 6, wherein the spiral segment has a first end that is connected to the base segment, and a second end that is free and is closer to the proximal end than the first end, along the longitudinal axis.

8. The method according to claim 6, and comprising:
inflating a balloon disposed over a portion of the distal end of the shaft, the balloon having a plurality of radiofrequency ablation electrodes arranged circumferentially over an external surface of the balloon; and
performing ablation using the ablation electrodes.

9. The method according to claim 8, wherein performing the ablation comprises performing pulmonary vein isolation.

10. The method according to claim 6, wherein advancing the reverse-lasso catheter comprises engaging an interior wall of a pulmonary vein.

11. A method of manufacturing a reverse-lasso catheter, the method comprising:
providing a shaft having a distal end and a proximal end, wherein the distal end of the shaft defines a longitudinal axis; and
disposing, at the distal end of the shaft, a reverse-lasso catheter comprising a base segment and a spiral segment that retrogrades in the proximal direction around the base segment as the spiral segment spirals about the longitudinal axis, wherein a plurality of sensing-electrodes disposed over the spiral segment.
